(19) United States
(12) Patent Application Publication
(10) Pub. No.: US 2022/0151549 A1
(43) Pub. Date: May 19, 2022

(54) CONTACTLESS MONITORING OF SLEEP ACTIVITIES AND BODY VITAL SIGNS VIA SEISMIC SENSING

(71) Applicant: University of Georgia Research Foundation, Inc., Athens, GA (US)

(72) Inventors: WenZhan SONG, Alpharetta, GA (US); Fangyu LI, Athens, GA (US); Jose CLEMENTE, Bogart, GA (US)

(21) Appl. No.: 17/427,528

(22) PCT Filed: Jan. 31, 2020
(86) PCT No.: PCT/US2020/016019

§ 371 Date: Jul. 30, 2021

Related U.S. Application Data
(60) Provisional application No. 62/799,825, filed on Feb. 1, 2019.

(51) Int. Cl. A61B 5/00; A61B 5/0205; A61B 5/024; A61B 5/08; A61B 5/11

(52) U.S. Cl. CPC A61B 5/4806; A61B 5/0205; A61B 5/02444; A61B 5/0816; A61B 5/11; A61B 5/4561; A61B 5/0002; A61B 5/743; A61B 5/746; A61B 5/7405; A61B 5/7455; A61B 5/7282; A61B 5/7475

(57) ABSTRACT

The present disclosure relates to a contactless sleep monitoring system and method for monitoring a plurality of characteristics of a subject based on vibration signals of a structure supporting the subject. The system can include a sensor that is coupled to the structure, but not in direct contact with the subject. A computing device in data communication with the sensor can obtain real-time sensor data from the sensor. The computing device can further analyze the sensor data to determine continuous and real-time measurements of characteristics of the subject where the characteristics can include a heart rate, a respiratory rate, a movement of the subject, and/or a posture of the subject. A user interface including a display of the determined measurements of the plurality of characteristics can be generated and displayed to a user.

### CROSS-REFERENCE TO RELATED APPLICATIONS

This application claims priority to, and the benefit of, co-pending U.S. provisional application entitled “CONTACTLESS MONITORING OF SLEEP ACTIVITIES AND BODY VITAL SIGNS VIA SEISMIC SENSING” having Ser. No. 62/779,825, filed on Feb. 1, 2019, which is hereby incorporated by reference in its entirety.

### BACKGROUND

Monitoring vital signs (heartbeat and respiration rate) is important to learn conditions for improvement and prevent potentially dangerous health threats like sleep apnea. Sleep monitoring is extremely important, even a life saver, for people with undiagnosed sleep apnea, which causes respiration and heart failures. In elder/special-needs community, monitoring the posture and the changes in posture during the time on bed are critical to determining long-periods of lack movement that can lead to health problems like eschars on the body. Other life-threating situations, such as, a fall off of a bed situation, for example, require a prompt detection and response. Vital signs like respiration status can be monitored by breathing apparatuses, while the heart rate is typically measured by wearable devices. However, those devices need body contact and are intrusive. Many people find these devices to be uncomfortable to wear or they forget to wear the devices before they sleep. Other devices can used for providing rapid assistance when people fall, but these devices require the person to actions that can only be taken while the person is conscience, such as, for example, pressing a button.

### BRIEF DESCRIPTION OF THE DRAWINGS

The foregoing will be apparent from the following more particular description of example embodiments of the present disclosure, as illustrated in the accompanying drawings in which like reference characters refer to the same parts throughout the different views. The drawings are not necessarily to scale, emphasis instead being placed upon illustrating embodiments of the present disclosure.

FIG. 1 illustrates an example of a contactless sleep monitoring system according to various embodiments of the present disclosure.

FIGS. 2A-2B illustrate examples of a contactless sleep monitoring system workflow according to various embodiments of the present disclosure.

FIG. 3 illustrates an example of a graphical representation illustrating sleep monitoring data according to various embodiments of the present disclosure.

FIG. 4 illustrates an example of a seismometer for sleep monitoring according to various embodiments of the present disclosure.

FIG. 5 illustrates an example graphical representation of obtained seismic data corresponding to before sleep respiration according to various embodiments of the present disclosure.

FIG. 6 illustrates an example graphical representation of obtained seismic data corresponding to during sleep respiration according to various embodiments of the present disclosure.

FIGS. 7A-7C illustrate examples of graphical representations showing the body motion and posture recognition data according to various embodiments of the present disclosure.

FIG. 8 illustrates a graphical representation of collected data indicating apnea according to various embodiments of the present disclosure.

FIG. 9 illustrates an example user interfaces associated with the contactless seismometer-based sleep monitoring system according to various embodiments.

FIG. 10 is a user interface illustration for smartphone devices with the statistics of the system according to various embodiments of the present disclosure.

FIG. 11 is a schematic block diagram that provides one example illustration of a computing environment according to various embodiments of the present disclosure.

### SUMMARY

Aspects of the present disclosure are related to a contactless sensor-based sleep monitoring system for monitoring characteristics of a subject, such as, for example, vital signs, postures, movements, falls of people/patients, etc. during sleep cycles using vibration signals from a structure supporting the subject (e.g., a bed).

In one aspect, among others, a system for monitoring a plurality of characteristics of a subject based on vibration signals of a structure supporting the subject, the system comprises a sensor coupled to the structure, at least one computing device in data communication with the sensor, and an application executable in the at least one computing device. When executed, the application can cause the at least one computing device to at least obtain real-time sensor data from the sensor, analyze the sensor data to determine continuous and real-time measurements of the plurality of characteristics of the subject, and generate a user interface comprising a display of the determined measurements of the plurality of characteristics; and render the user interface via a display. The plurality of characteristics can comprise at least one of: a heart rate, a respiratory rate, a movement of the subject, or a posture of the subject.

In various aspects, the application can further cause the at least one computing device to at least determine the heart rate based at least in part on a local maxima statistics method. In various aspects, the application can cause the at least one computing device to at least determine the respiratory rate by estimating an amplitude, frequency and phase associated with the sensor data. In various aspects, the application can cause the at least one computing device to at least detect the posture of the subject according to an instantaneous amplitude of respiration extracted from sensor data. In various aspects, the application can cause the at least one computing device to at least detect an event based at least in part on at least one of the heart rate, the respiratory rate, the posture of a subject, or a movement of the subject. In various aspects, the event can comprise at least one of a fall of the subject, the heart rate being outside a predefined range, the respiratory rate being outside a predefined range, or a change in the posture. In various aspects, the application can cause the at least one computing device to at least generate an alert in response to the detected event. In various aspects, the alert is at least one of an auditory or visual or vibratory alert. In various aspects, the at least one computing device is in communication with a smart device configured to communicate with a third party, and generating the alert further comprises instructing the smart device to send a communication with the third party. In various aspects, the sensor is not in direct contact with the subject.

In another aspect, among others, a method for monitoring a subject, comprises receiving, via at least one computing device, sensor data from a sensor coupled to structure, the sensor data corresponding to one or more vibrations of the structure, analyzing, via the at least one computing device, the seismic data to determine a heart rate, a respiratory rate, a movement and a posture of a subject supported by the structure, generating, via the at least one computing device, a user interface comprising the heart rate, the respiratory rate, and the posture of the subject, and rendering, via the at least one computing device, the user interface via a display.

In various aspects, the method further comprises updating the user interface to include at least one of: an updated heart rate, an updated respiratory rate, an updated movement detection, an updated alert or an updated posture. In various aspects, the method further comprises determining the heart rate based at least in part on a local maxima statistics method. In various aspects, the method further comprises determining the respiratory rate by estimating an amplitude, frequency and phase associated with the sensor data. In various aspects, the method further comprises detecting the posture of the subject according to an instantaneous amplitude of respiration extracted from sensor data. In various aspects, the method further comprises detecting an event based at least in part on at least one of the heart rate, the respiratory rate, the posture of a subject, or a movement of the subject. In various aspects, the method the event comprises at least one of a fall, the heart rate being outside a predefined range, the respiratory rate being outside a predefined range, or a change in the posture. In various aspects, the method further comprising generating an alert in response to the detected event. In various aspects, the alert is at least one of an auditory or visual or vibratory alert. In various aspects, the at least one computing device is in communication with a smart device configured to communicate with a third party, and generating the alert further comprises instructing the smart device to send a communication with the third party.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DETAILED DESCRIPTION

The present disclosure relates to a contactless sensor-based sleep monitoring system for monitoring characteristics of a subject such as, for example, vital signs, postures, movements and falls of people/patients during sleep cycles using vibration signals from a structure supporting the subject (e.g., a bed, a chair, etc.). Monitoring sleep status is important to learn health condition and life-threating events. According to various embodiments of the present disclosure, the contactless sleep monitoring system is configured to monitor the heart rate, respiratory rate, body movement, body posture, and fall off bed of a subject. To effectively monitor sleep status, an innovative local maxima statistics based approach and an instantaneous property based method are used to estimate heart and respiratory rates, respectively. These methods are more robust and stable compared to known methods. In addition, instantaneous properties of obtained seismic data can be used to detect body movement and posture identification. Seismic signals related to fall downs are using for detecting fall off from bed.

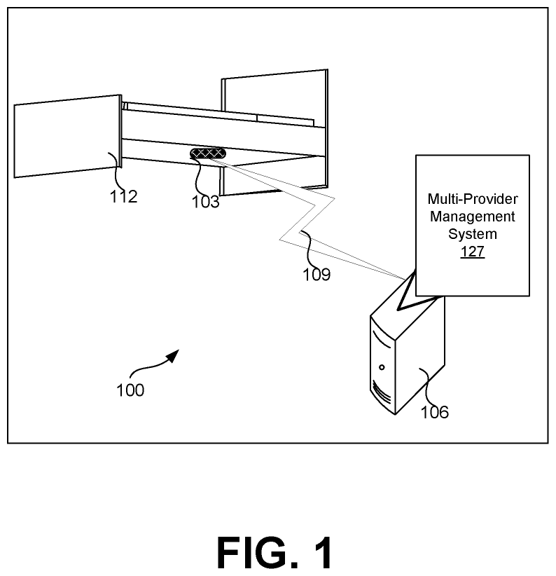

FIG. 1 illustrates an example of the contactless sleep monitoring system 100 according to various embodiments of the present disclosure. According to various embodiments, the contactless sleep monitoring system of the present disclosure comprises a sensor 103 (e.g., seismometer sensor) in data communication with a computing device 106 via a wireless or wired connection 109. The sensor 103 can be attached to a structure 112 (e.g., bed frame) that is associated with a subject. The sensor 103 can be attached to the structure 112 such that it is not in direct contact with the subject. For example, the sensor 103 can be positioned on the underside of a bed away from the top side of the bed where the subject lays down. The computing device 106 comprises at least a seismic tracking application 115 that, when executed, by the computing device, is configured to obtain sensor data from the sensor 103 and analyze the sensor data to identify characteristics of a subject. The characteristics can include heart rate, respiratory rate, body posture, movement, fall off bed, and/or other features associated with the subject.

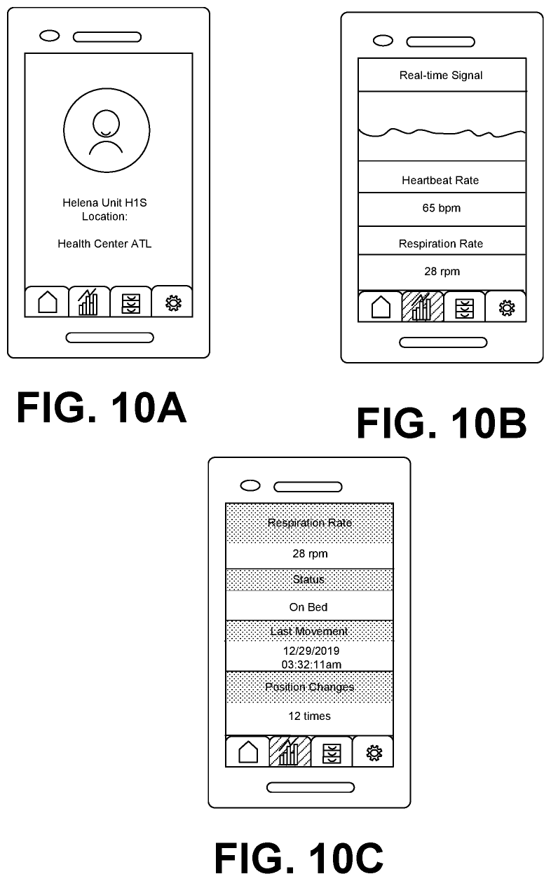

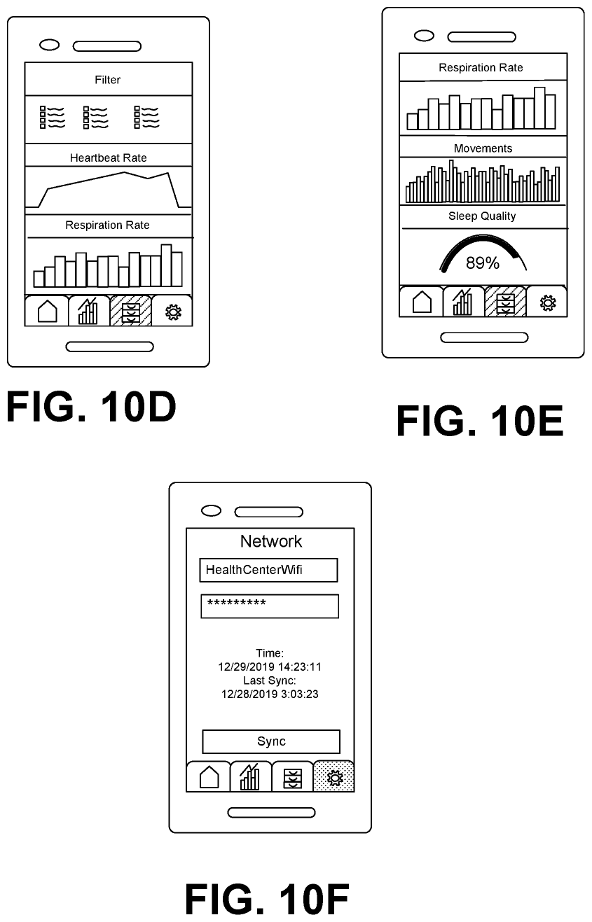

According to various embodiments, the contactless sleep monitoring system 100 of the present disclosure is configured to generate one or more user interfaces (FIG. 10) that include information associated with a subject's sleep quality and status. For example, the one or more user interfaces can include a seismic signal, a heart rate, a respiration rate, a posture detection, a number of alerts (e.g., fall of bed alerts), a current position, and/or other information. The one or more user interfaces can be updated periodically or randomly.

In some embodiments, the one or more user interfaces can be rendered upon a display of the computing device 106 of the contactless sleep monitoring system 100. In other embodiments, the computing device 106 can be in data communication with a client device (e.g., mobile device) (not shown) over a network. The client device can be configured to render the one or more user interfaces on a display of the client device. In some embodiments, the client device can obtain sensor data from the computing device and can generate the one or more user interfaces using the obtained sensor data.

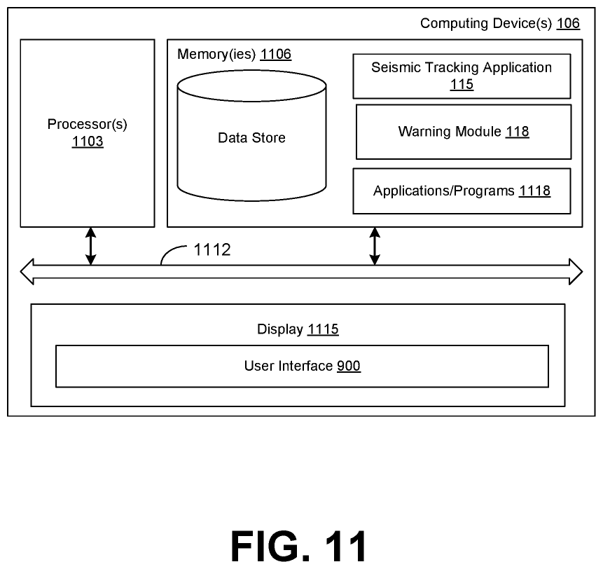

In some embodiments, the contactless sleep monitoring system 100 can detect an event and comprise a warning module 118 (FIG. 11) configured to generate an alert in an instance in which a particular event is detected. In some embodiments, the warning module 118 is implemented as part of the computing device 106 of the sleep monitoring system 100. For example, the warning system can comprise an application executable in the computing device 106. In other embodiments, the warning module 118 can be separate from the computing device 106 and can be data communication with the computing device 106 over a network.

A detected event can comprise a fall, a heart rate being outside a predefined range, a respiratory rate being outside a predefined range, and/or other event. Upon detection of an event, the warning module 118 can cause an alert to be generated to notify the subject and/or other entities about the event. In some embodiments, the alert can comprise a visual and/or auditory alert. In other embodiments, the warning module 118 can be coupled to a communication device that can notify an emergency entity (e.g., hospital, doctor, 911, etc.) and/or other entity. For example, the warning module 118 can be in communication with a smart device (e.g., smart speaker) that is capable of making an emergency call and/or otherwise notifying emergency personal and/or other persons.

Seismometers, including geophones and accelerometers, have been widely used in geophysical and civil engineering applications. Recently, new applications for smart environments have been explored, such as ambient vibration for building occupancy estimation, floor vibration for indoor person localization, bed vibration for heart beating and breathing rate monitoring, etc.

The target sleep vital signal measurements are heartbeat and respiratory rates, whose timing and durations are important. However, the traditional harmonic analysis is not suitable for bio-signal processing and analysis because of the data non-stationary nature. Nowadays, as an important tool for non-stationary signal analysis, oscillatory analysis has been widely applied. The hypothesis of oscillatory analysis is that a signal contains a few principle and minor components with different oscillation patterns. For example, in one known process, both respiratory and cardiac rhythms were extracted from PPG (photoplethysmogram) signals using an oscillatory mode decomposition.

According to various embodiments of the present disclosure, a local maxima statistics method estimates the heart rate, and instantaneous property from oscillatory analysis is used to characterize the respiration rate. Different from known methods, the strict periodicity property of the heartbeat is not required and thus the present disclosure is more robust. Further, instead of using the envelope based respiratory rate estimation of known methods, the instantaneous property-based method is designed for robust and stable estimation.

According to various embodiments, algorithms for detecting body movements and sleep posture changes are disclosed. Evaluations demonstrate that the contactless sleep monitoring system 100 of the present disclosure, which is non-intrusive and contactless, is effective for monitoring sleep status and quality and detecting apnea phenomenon.

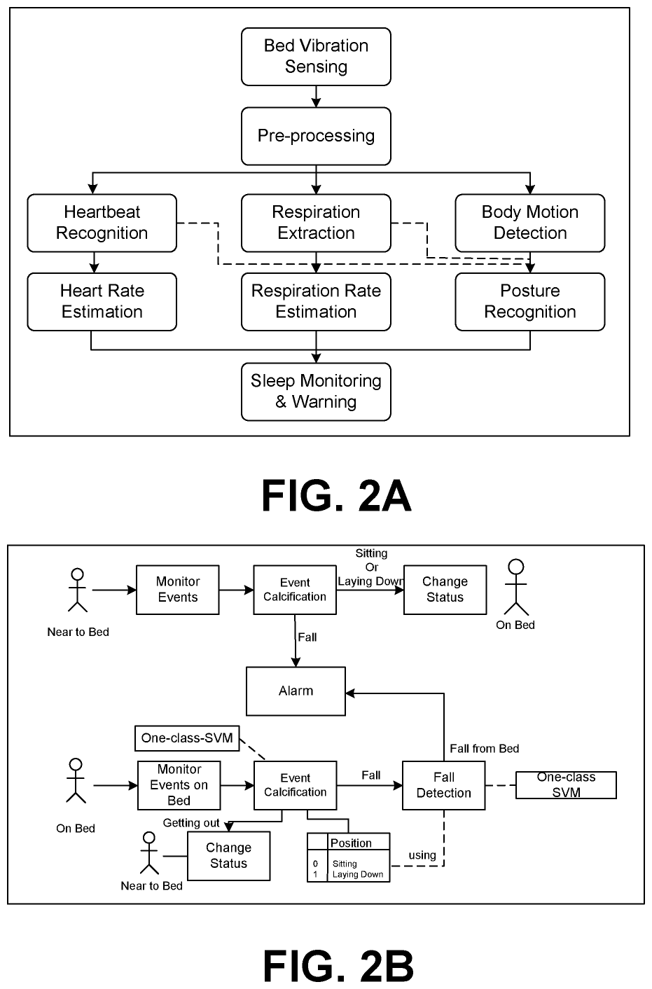

For sleep monitoring, heart and respiratory rates, as well as body movement and sleep posture are important parameters. The present disclosure provides different algorithms for parameter estimation and monitoring. FIGS. 2A-2B provide example sleep monitoring system workflows according to various embodiments of the present disclosure.

For elder/special-needs community, the fall off detection is also an important feature for timely assisting people. The present disclosure provides algorithms for fall off detection using the seismic/vibration data. FIG. 2B provides an example of fall off detection workflow according to various embodiments of the present disclosure.

### Heart Rate Estimation

Estimating heart rate BPMₕ directly from the data spectrum is not accurate because the heartbeat waveform is not strictly periodical in reality. BPMₕ denotes beats per minute for heart rate and BPMᵣ denotes breaths per minute for respiratory rates. To avoid periodicity dependency, a novel local maxima statistics method is disclosed to address this challenge.

Since a heartbeat generates one peak on the recorded seismometer data s(t), the point (t,s(t)) is defined as the local maximum within an interval Iₕ if s(t)≥S(Z) for every

$$Z ∈ ( t - \frac{{I}_{h}}{2} , t + \frac{{I}_{h}}{2} ) ,$$

where Iₕ is initialized according to the heartbeat frequency range. In addition, the heartbeat strength (amplitude) can also be a constraint during the local maxima search. However, even with filtering and autocorrelation operations, the heartbeat recognition results are not stable and can be influenced by interferences.

To solve instabilities, a novel empirical truncated statistics analysis method is disclosed to estimate BPMₕ. When local maxima are obtained, there are falsely picked peaks and some missing peaks. Those falsely picked peaks result in smaller period estimation, while the missed peaks lead to larger estimation results. Here, X is the interval between two sequential picked peaks. The heartbeat period within

$$( t - \frac{{I}_{h}}{2} , t + \frac{{I}_{h}}{2} )$$

is estimated as a truncated averages:

$$E ( X \vert {F}^{- 1} ( a ) < X \leq {F}^{- 1} ( b ) ) = \frac{{\int }_{a}^{b} xg ( x ) dx}{F ( b ) - F ( a )} , \\ where , \\ g ( x ) = f ( x ) for {F}^{- 1} ( a ) < X \leq {F}^{- 1} ( b ) ; \\ g ( x ) = 0 , everywhere else ; \\ {F}^{- 1} ( p ) = inf \{ x \text{:} F ( x ) \geq p \} . \qquad ( 1 )$$

The lower and upper bounds (a and b) are determined based on the local maxima detection performance. In the examples of the present disclosure, 0.1 and 0.9 are chosen, respectively.

### Respiratory Rate Estimation

Commodity seismometers are insensitive to lower frequency measurements (usually lower than 0.3 Hz), thus the respiratory rate BPMᵣ cannot be directly observed from seismic data. An amplitude-modulation approach has been previously proposed to use the envelope to estimate carrier frequency. However, the amplitude modulation of the recorded seismometer signal is not stable. According to experiments, the lower and upper envelopes usually show different behavior, so it is difficult to use the amplitude modulation methods for reliable estimation.

According to various embodiments of the present disclosure, a novel signal configuration model formulates the relation among seismic data, heartbeat and respiration components. Then, oscillatory analysis technique synchrosqueezed wavelet packet transform (SSWPT) is used to extract the instantaneous properties of the respiration mode. In oscillatory analysis, a non-linear and non-stationary wave-like signal s(t) is defined as a superposition of several oscillatory components:

s(t)=Σ^{K}ₖ₌₁αₖ(t)e^{2πiN}^{_{kϕk}}^{(t)+n(t)} (2)

where, αₖ(t) is the instantaneous amplitude, N_{kϕk}(t) is the instantaneous phase, N_{kϕ′k}(t) is the instantaneous frequency, and n(t) is the noise contamination. In the experiment, α₀(t) and N_{0ϕ′0}(t) correspond to the wanted respiration component.

The instantaneous properties (amplitude, frequency and phase) in Equation 2 are not known and can be are estimated via SSWPT. Suppose Wₛ(ξ, t) is the wavelet transform of a 1D wave-like component. It was proved that the instantaneous frequency information function

$${v}_{s} ( ξ , t ) = \frac{{\partial }_{t} {W}_{s} ( ξ , t )}{2 π {iW}_{s} ( ξ , t )}$$

is able to approximate Nϕ′(t). Hence, the SSWPT is used to obtain a sharpened instantaneous property estimation compared to the traditional methods. When the instantaneous amplitude (IA) of respiration is extracted, the respiratory rate can be easily obtained.

### Body Motion/Movement and Posture Recognition

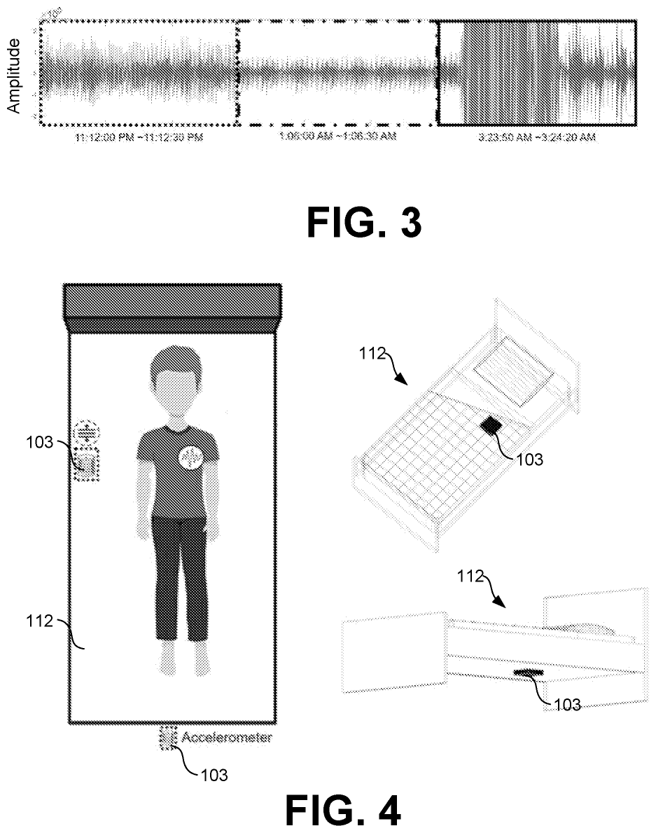

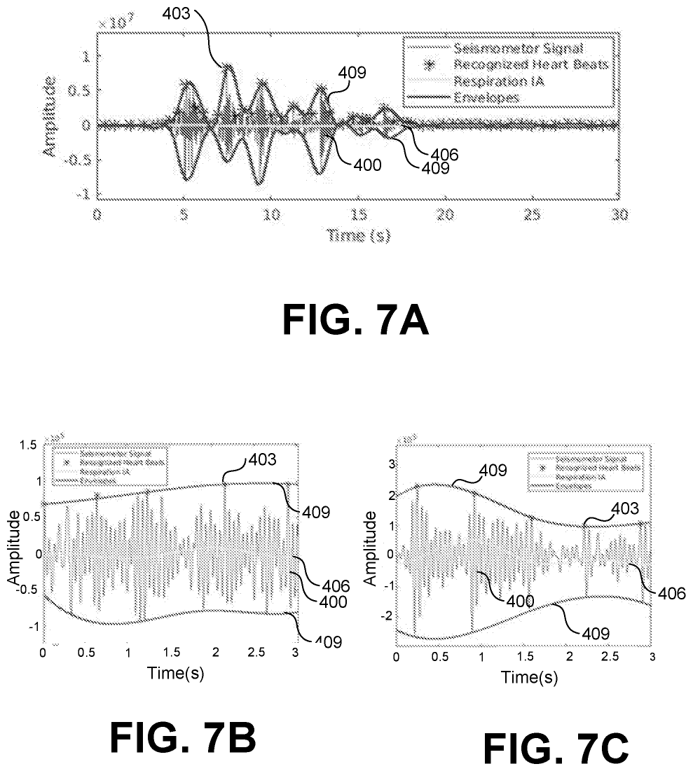

In previous experiments, all the subjects lay down on their backs. However, the sleep posture influences the recorded data quality and property. FIG. 3 and FIG. 7 illustrate graphical representations showing that the body movement generates strong signal (107 amplitude) while the respiration and heartbeat show amplitude about 105. In particular, FIG. 3 illustrates sleep monitoring data where sensor data are recorded before sleep (e.g. 11 pm), during sleep (e.g., 1 AM) as well as before and after sleeping posture changes (3 AM). Thus, based on the dramatic energy change, the body movement can be recognized using a local thresholding method:

$${Tr}_{m} = \{ \begin{matrix}1 , & if s ( t ) \geq λ max ( s ( t ) ) , Z ∈ ( t - τ , t ) , \\ 0 , & otherwise\end{matrix} \qquad ( 3 )$$

where, λ is a threshold coefficient and τ is the time lag.

In addition, the IA of respiration usually changes after the body movement is detected, which probably means the sleep posture has changed. Thus, the posture changes can also be detected by applying Equation 3 to IA, but with a different λ.

### Fall Off Bed Recognition

According to various embodiments of the present disclosure, a novel “fall off bed” detection is implemented to recognize fall off and send alarms. High amplitudes and motion events on bed are classified as fall or not fall. This is done using a one-class support vector machine (SVM). When the occurred event is classified as fall down, an alarm is sent to a device/application for notification.

### EXPERIMENTS

The sleep monitoring system 100 of the present disclosure is designed to continuously monitor sleep signals. In the following experiment, the seismometer is attached to a bed frame, which is non-intrusive and non-contact to human body. A computing device 106 is connected with the sensor 103 (e.g. seismometer) for real-time data processing. FIG. 4 illustrates an example of the contactless sleep monitoring system 100 system according to various embodiments of the present disclosure. In particular, FIG. 4 illustrates a sensor 103 attached to a bed side 112 and coupled to a computing device 106. FIG. 3 illustrates non-limiting examples showing the sensor 103 mounted on the bed. For example, FIG. 3 illustrates an example of the sensor 103 mounted to the underside of the bed 112. FIG. 3 illustrates another example of the sensor 103 mounted on the top side of the bed 112. In various embodiments, the sensor 103 can comprise a seismometer that is naturally a second-order high-pass filter and its general syntonic frequency can be 8 Hz. The vertical channel signal is used in our experiments.

### Body Parameter Monitoring

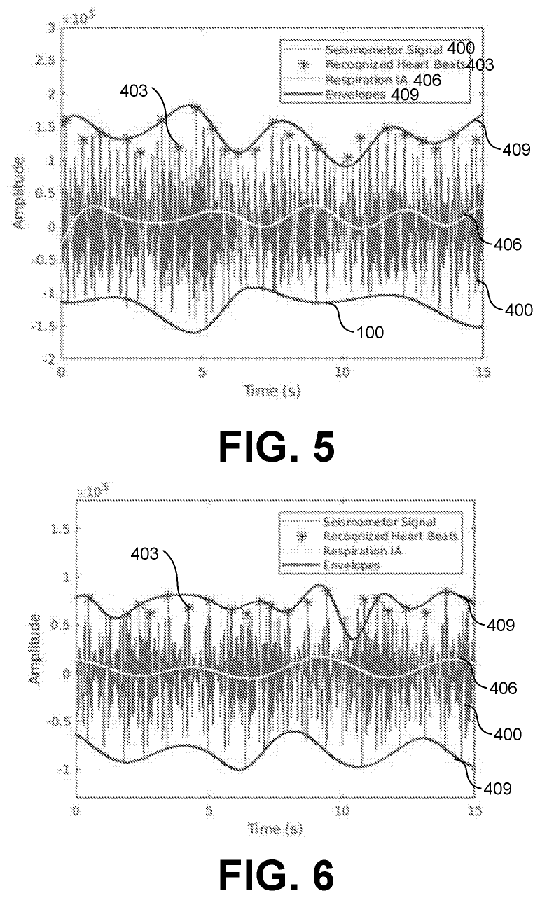

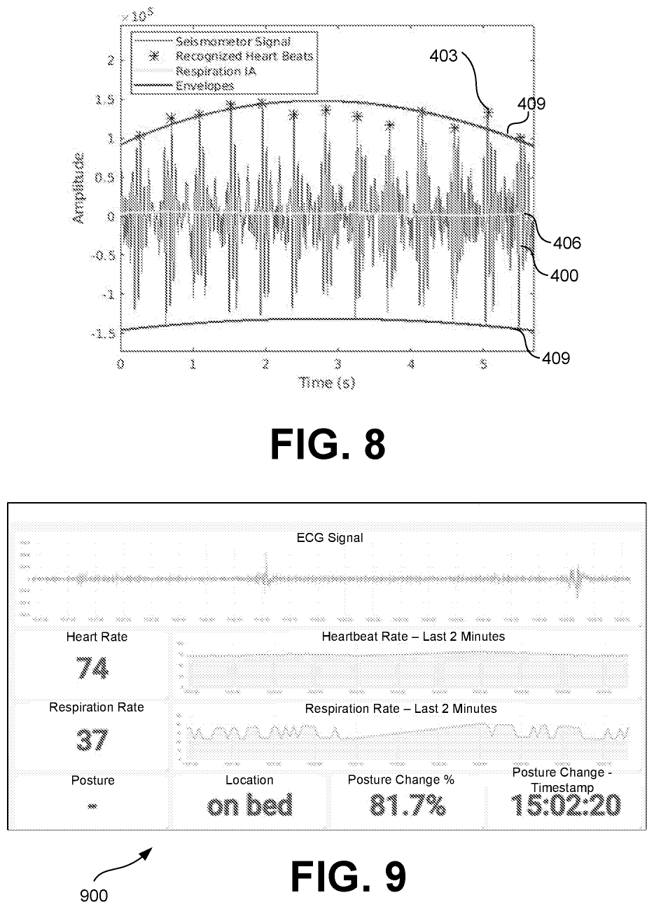

FIG. 3 shows three recorded segments: before sleep, normal sleep and body movement, which are extracted from an eight hour sleep monitoring data set of a human subject. Using the local maxima search method, the recognized heartbeats are shown in FIG. 5, FIG. 6, FIG. 7, and FIG. 8. In particular, FIG. 5 illustrates an example of a graphical representation of the before sleep example. In particular, FIG. 5 illustrates an example of the before sleep segment from FIG. 3 showing the recognized heartbeats, envelopes and respiration IA.

FIG. 6 illustrates an example of a graphical representation of the during sleep example of FIG. 3 according to various embodiments. In particular, FIG. 6 illustrates an example of the during sleep segment from FIG. 3 showing the recognized heartbeats, envelopes and respiration IA. When compared to the before sleep representation of FIG. 54, the respiration is slower and the IA is weaker in FIG. 6.

FIGS. 7A-7C illustrate examples of graphical representations showing the body motion and posture recognition data according to various embodiments of the present disclosure. In particular, 7A illustrates body movement as shown by the strong amplitudes. 7B illustrates the body motion before the movement and 7B illustrates the body motion after the movement. As shown in FIG. 7C, the IA changes, which indicates a posture change.

FIG. 8 illustrates a graphical representation of collected data indicating apnea (no respiration for a period of time) according to various embodiments of the present disclosure. In particular, FIG. 8 illustrates a seismometer signal 400, recognized heartbeats 403, envelopes 409, and respiration instantaneous amplitude 406.

According to the BPMₕ estimation in Equation 1, the subject has a 90 BPMₕ before sleep (FIG. 5) and a 75 BPMₕ during sleep (FIG. 6), which are validated by the smart watch wore.

In order to compare with the envelope-based method, the instantaneous amplitude (IA) of the respiration component α₀(t) and the upper and lower envelopes are plotted as shown in FIG. 5. From the envelopes the BPMᵣ can be estimated, however, the upper and lower envelopes do not always have the same periodicity. In addition, the envelope extraction is sensitive to parameters, leading to that the respiration rate estimation have been previously constrained by the predefined parameters. The middle curve in FIG. 5 is the IA of the extracted respiration component from SSWPT. According to the spectral analysis, the subject's respiratory rate is about 15.6 BPMᵣ before sleep (FIG. 5), which is very close to the ground truth measured by a stop watch. And the BPMᵣ during sleep is 12.4.

### Sleep Quality and Posture

According to various embodiments of the present disclosure, the sleep monitoring system of the present disclosure is configured to detect the sleep quality and posture. If the subject has a lot of movements and motions, it means the sleep quality is not good. FIG. 3 shows a late night data at 3 AM within a solid frame. FIGS. 7A-7C show the signal is too strong (100 times larger) compared with just heartbeats and respiration. Using the amplitude anomalies, the body motions and movements can be recorded and analyzed for sleep quality determination.

In FIG. 3 and FIGS. 7A-7C, the average peak amplitudes are about 1×10⁵ before body movement, but after the movement peak amplitudes become around 1.5˜3×10⁵, which means the respiration is stronger when the subject changes a posture. According to various embodiments, the oscillatory components can be connected with the sleep postures. The new information about the sleep status will make a more detailed sleep analysis report possible, which can provide more health advice. In addition, according to various embodiments, a feature learning approach such as machine learning can be used to identify the different postures. For example, the latter part of the new posture signal shows double peaks for one heartbeat, which is different with that before the posture change. This information can be used to learn about the subject and how the subject's body reacts to posture changes.

### Apnea Detection and Alert

Apnea or apnoea is suspension of breathing. During apnea, there is no movement of the muscles of inhalation, and the volume of the lungs initially remains unchanged. Depending on how blocked the airways are (patency), there may or may not be a flow of gas between the lungs and the environment. This can be dangerous situation. FIG. 8 shows a 5 s seismometer signal record when a subject lay on a bed holding breath for at least ten (10) seconds. The recognized heartbeats 403, envelopes 409 as well as the IA of the respiration 406 are also shown. It is obvious that the respiratory rate is too low. In this situation, an embedded warning module 118 connected with a commercial smart home system (not shown) can to make an emergency call or notify other people.

The sleep monitoring system 100 of the present disclosure is non-intrusive and contactless, showing great potentials for sleep quality and status monitoring. Viewing the respiration and heartbeat are different rhythms of human body, oscillatory components can be extracted to estimate those body parameters. A novel local maxima statistics method and a SSWPT based instantaneous property analysis approach are designed to estimate heart and respiratory rate. The experiments demonstrate that the oscillatory analysis is promising for time series bio-signal data analysis. The extracted oscillatory components help extract the signal rhythms and useful information on amplitude and frequency for not only heart/respiration rate estimation, but also body movement and posture identification. In addition, the system of the present disclosure can detect a user's slight activities such as snores during sleep. In addition, using machine learning and deep learning models, a more sophisticated sleep monitoring system can be developed to accurately detect a person's sleep stage and evaluate the sleep quality. In particular, machine learning and deep learning models can be used to detect and classify the collected information associated with a person's sleep stage and sleep quality.

Turning now to FIG. 9, shown is an example user interface 900 associated with the sleep monitoring tracking system 100 according to various embodiments. According to various embodiments, the user interface 900 can include a seismic signal 400, a heart rate, a respiration rate, a posture detection, a number of alerts (e.g., fall of bed alerts), a current position, and/or other information about the subject.

The seismic signal 400 can comprise sensor data obtained from the sensor 103 on a bed frame 112 and/or other structure. The visualization of the seismic signal 400 can be updated periodically. For example, the signal can be updated every five (5) seconds. The update rate can be adjusted by a user as can be appreciated. According to various embodiments, a user can interact with the user interface 900 to search between different date ranges to visualize how the signal at that moment.

The heart rate provided in the user interface 900 can show the heart rate when the person lays on the bed. The number is shown after the analysis of the heart signal. The respiration rate is extracted after the person lays on the bed and the heart signal is obtained. The posture detection elements of the user interface show the posture the person is lying on the bed. It has four status: “Right”, “Left”, “Back” or “Chest” depending on the actual body position. Also, if the person is moving, the displayed message is “Movement”.

The number of alerts can include a number of fall off bed or alerts. For example, when a person falls off the bed, the system 100 can register an alert and send a message to a smart device, for example, for alerts. These episodes are registered in this element card. The current position of the person can include a status associated with whether the subject is “OFF BED” or when the subject is “ON BED”.

FIGS. 10A-10F illustrate example user interfaces 900 (e.g., 900a-900f) which can be displayed on mobile devices that require smaller screen space, according to various embodiments of the present disclosure. Location of the sensor and patient, real-time signal, heart rate, respiration rate, status, last movements, position changes, historical analytics, and settings can be displayed while using the application on a smartphone.

With reference now to FIG. 11, shown is one example of at least one computing device 106 (e.g., an interfacing device, central server, server, or other network device) that performs various functions of the seismic data analysis algorithms in accordance with various embodiments of the present disclosure. Each computing device 106 includes at least one processor circuit, for example, having a processor 1103 and a memory 1106, both of which are coupled to a local interface. To this end, each computing device 106 may be implemented using one or more circuits, one or more microprocessors, microcontrollers, application specific integrated circuits, dedicated hardware, digital signal processors, microcomputers, central processing units, field programmable gate arrays, programmable logic devices, state machines, or any combination thereof. The local interface 1112 may comprise, for example, a data bus with an accompanying address/control bus or other bus structure as can be appreciated. Each computing device 106 can include a display 1115 for rendering of generated graphics such as, e.g., a user interface 900 and an input interface such, e.g., a keypad or touch screen to allow for user input. In addition, each computing device 106 can include communication interfaces (not shown) that allows each computing device to communicatively couple with other communication devices. The communication interfaces may include one or more wireless connection(s) such as, e.g., Bluetooth or other radio frequency (RF) connection and/or one or more wired connection(s).

Stored in the memory 1106 are both data and several components that are executable by the processor 1103. In particular, stored in the memory and executable by the processor are seismic tracking application(s) 115, warning module 118, and/or other applications 1118. Seismic tracking applications 115 can include applications that interact with the sensor 103 attached to a structure and detect entity characteristics associated with sleep quality and/or posture. The warning module 118 can include applications that can generate alerts to notify other individuals and/or emergency entities in response to a detected event. It is understood that there may be other applications 1118 that are stored in the memory 1106 and are executable by the processor 1103 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Delphi®, Flash®, LabVIEW® or other programming languages.

A number of software components are stored in the memory 1106 and are executable by the processor 1103. In this respect, the term “executable” means a program file that is in a form that can ultimately be run by the processor 1103. Examples of executable programs may be, for example, a compiled program that can be translated into machine code in a format that can be loaded into a random access portion of the memory and run by the processor, source code that may be expressed in proper format such as object code that is capable of being loaded into a random access portion of the memory and executed by the processor 1103, or source code that may be interpreted by another executable program to generate instructions in a random access portion of the memory 1106 to be executed by the processor 1103, etc. An executable program may be stored in any portion or component of the memory 1106 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1106 is defined herein as including both volatile and nonvolatile memory and data storage components. Volatile components are those that do not retain data values upon loss of power. Nonvolatile components are those that retain data upon a loss of power. Thus, the memory 1106 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1103 may represent multiple processors and the memory 1106 may represent multiple memories that operate in parallel processing circuits, respectively. In such a case, the local interface 1112 may be an appropriate network that facilitates communication between any two of the multiple processors 1103, between any processor 1103 and any of the memories 1106, or between any two of the memories, etc. The local interface 1112 may comprise additional systems designed to coordinate this communication, including, for example, performing load balancing. The processor 1103 may be of electrical or of some other available construction.

Although the seismic tracking application(s) 115, warning module 118, other applications 1118 and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

Also, any logic or application described herein, including seismic tracking application(s) 115, and warning module 118, that comprises software or code can be embodied in any non-transitory computer-readable medium for use by or in connection with an instruction execution system such as, for example, a processor in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

In addition to the foregoing, the various embodiments of the present disclosure include, but are not limited to, the embodiments set for in the following clauses.

Clause 1. A system for monitoring a plurality of characteristics of a subject based on vibration signals of a structure supporting the subject, the system comprising: a sensor coupled to the structure; at least one computing device in data communication with the sensor; and an application executable in the at least one computing device, wherein when executed, the application causes the at least one computing device to at least: obtain real-time sensor data from the sensor; analyze the sensor data to determine continuous and real-time measurements of the plurality of characteristics of the subject, the plurality of characteristics comprising at least one of: a heart rate, a respiratory rate, a movement of the subject, or a posture of the subject; generate a user interface comprising a display of the determined measurements of the plurality of characteristics; and render the user interface via a display.

Clause 2. The system of clause 1, wherein, when executed, the application further causes the at least one computing device to at least determine the heart rate based at least in part on a local maxima statistics method.

Clause 3. The system of any one of clauses 1 or 2, wherein, when executed, the application further causes the at least one computing device to at least determine the respiratory rate by estimating an amplitude, frequency and phase associated with the sensor data.

Clause 4. The system of any one of clauses 1 to 3, wherein, when executed, the application further causes the at least one computing device to at least detect the posture of the subject according to an instantaneous amplitude of respiration extracted from sensor data.

Clause 5. The system of any one of clauses 1 to 4, wherein, when executed, the application further causes the at least one computing device to at least detect an event based at least in part on at least one of the heart rate, the respiratory rate, the posture of a subject, or a movement of the subject.

Clause 6. The system of clause 5, wherein the event comprises at least one of a fall of the subject, the heart rate being outside a predefined range, the respiratory rate being outside a predefined range, or a change in the posture.

Clause 7. The system of any one of clauses 5 or 6, wherein, when executed, the application further causes the at least one computing device to at least generate an alert in response to the detected event.

Clause 8. The system of clause 7, wherein the alert is at least one of an auditory or visual or vibratory alert.

Clause 9. The system of any one of clauses 7 or 8, wherein the at least one computing device is in communication with a smart device configured to communicate with a third party, and generating the alert further comprises instructing the smart device to send a communication with the third party.

Clause 10. The system of any one of clauses 1 to 9, wherein the sensor is not in direct contact with the subject.

Clause 11. A method for monitoring a subject, comprising receiving, via at least one computing device, sensor data from a sensor coupled to structure, the sensor data corresponding to one or more vibrations of the structure; analyzing, via the at least one computing device, the seismic data to determine a heart rate, a respiratory rate, a movement and a posture of a subject supported by the structure; generating, via the at least one computing device, a user interface comprising the heart rate, the respiratory rate, and the posture of the subject; and rendering, via the at least one computing device, the user interface via a display.

Clause 12. The method of clause 11, further comprising updating the user interface to include at least one of: an updated heart rate, an updated respiratory rate, an updated movement detection, an updated alert or an updated posture.

Clause 13. The method of any one of clauses 11 or 12, further comprising determining the heart rate based at least in part on a local maxima statistics method.

Clause 14. The method of any one of clauses 11 to 13, further comprising determining the respiratory rate by estimating an amplitude, frequency and phase associated with the sensor data.

Clause 15. The method of any one of clauses 11 to 14, further comprising detecting the posture of the subject according to an instantaneous amplitude of respiration extracted from sensor data.

Clause 16. The method of any one of clauses 11 to 15, further comprising detecting an event based at least in part on at least one of the heart rate, the respiratory rate, the posture of a subject, or a movement of the subject.

Clause 17. The method of clause 16, wherein the event comprises at least one of a fall, the heart rate being outside a predefined range, the respiratory rate being outside a predefined range, or a change in the posture.

Clause 18. The method of any one of clauses 16 to 17, further comprising generating an alert in response to the detected event.

Clause 19. The method of clause 18, wherein the alert is at least one of an auditory or visual or vibratory alert.

Clause 20. The method of any one of clauses 18 or 19, wherein the at least one computing device is in communication with a smart device configured to communicate with a third party, and generating the alert further comprises instructing the smart device to send a communication with the third party.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A system for monitoring a plurality of characteristics of a subject based on vibration signals of a structure supporting the subject, the system comprising:
a sensor coupled to the structure;
at least one computing device in data communication with the sensor; and
an application executable in the at least one computing device, wherein when executed, the application causes the at least one computing device to at least:
obtain real-time sensor data from the sensor;
analyze the sensor data to determine continuous and real-time measurements of the plurality of characteristics of the subject, the plurality of characteristics comprising at least one of: a heart rate, a respiratory rate, a movement of the subject, or a posture of the subject;
generate a user interface comprising a display of the determined measurements of the plurality of characteristics; and
render the user interface via a display.

2. The system of claim 1, wherein, when executed, the application further causes the at least one computing device to at least determine the heart rate based at least in part on a local maxima statistics method.

3. The system of claim 1, wherein, when executed, the application further causes the at least one computing device to at least determine the respiratory rate by estimating an amplitude, frequency and phase associated with the sensor data.

4. The system of claim 1, wherein, when executed, the application further causes the at least one computing device to at least detect the posture of the subject according to an instantaneous amplitude of respiration extracted from sensor data.

5. The system of claim 1, wherein, when executed, the application further causes the at least one computing device to at least detect an event based at least in part on at least one of the heart rate, the respiratory rate, the posture of a subject, or a movement of the subject.

6. The system of claim 5, wherein the event comprises at least one of a fall of the subject, the heart rate being outside a predefined range, the respiratory rate being outside a predefined range, or a change in the posture.

7. The system of claim 5, wherein, when executed, the application further causes the at least one computing device to at least generate an alert in response to the detected event.

8. The system of claim 7, wherein the alert is at least one of an auditory or visual or vibratory alert.

9. The system of claim 7, wherein the at least one computing device is in communication with a smart device configured to communicate with a third party, and generating the alert further comprises instructing the smart device to send a communication with the third party.

10. The system of claim 1, wherein the sensor is not in direct contact with the subject.

11. A method for monitoring a subject, comprising
receiving, via at least one computing device, sensor data from a sensor coupled to structure, the sensor data corresponding to one or more vibrations of the structure;
analyzing, via the at least one computing device, the seismic data to determine a heart rate, a respiratory rate, a movement and a posture of a subject supported by the structure;
generating, via the at least one computing device, a user interface comprising the heart rate, the respiratory rate, and the posture of the subject; and
rendering, via the at least one computing device, the user interface via a display.

12. The method of claim 11, further comprising updating the user interface to include at least one of: an updated heart rate, an updated respiratory rate, an updated movement detection, an updated alert or an updated posture.

13. The method of claim 11, further comprising determining the heart rate based at least in part on a local maxima statistics method.

14. The method of claim 11, further comprising determining the respiratory rate by estimating an amplitude, frequency and phase associated with the sensor data.

15. The method of claim 11, further comprising detecting the posture of the subject according to an instantaneous amplitude of respiration extracted from sensor data.

16. The method of claim 11, further comprising detecting an event based at least in part on at least one of the heart rate, the respiratory rate, the posture of a subject, or a movement of the subject.

17. The method of claim 16, wherein the event comprises at least one of a fall, the heart rate being outside a predefined range, the respiratory rate being outside a predefined range, or a change in the posture.

18. The method of claim 16, further comprising generating an alert in response to the detected event.

19. The method of claim 18, wherein the alert is at least one of an auditory or visual or vibratory alert.

20. The method of claim 18, wherein the at least one computing device is in communication with a smart device configured to communicate with a third party, and generating the alert further comprises instructing the smart device to send a communication with the third party.
